(19) United States
(12) Patent Application Publication
(10) Pub. No.: US 2019/0013089 A1
(43) Pub. Date: Jan. 10, 2019

(54) METHOD AND SYSTEM TO IDENTIFY DOMINANT PATTERNS OF HEALTHCARE UTILIZATION AND COST-BENEFIT ANALYSIS OF INTERVENTIONS

(71) Applicant: KONINKLIJKE PHILIPS N.V., EINDHOVEN (NL)

(72) Inventor: Ushanandini RAGHAVAN, Lexington, MA (US)

(21) Appl. No.: 16/066,368

(22) PCT Filed: Dec. 28, 2016
(86) PCT No.: PCT/EP2016/082789

§ 371 Date: Jun. 27, 2018

Related U.S. Application Data
(60) Provisional application No. 62/272,209, filed on Dec. 29, 2015.

(51) Int. Cl. G16H 15/00; G16H 10/60; G06Q 40/08

(52) U.S. Cl. CPC G16H 15/00; G06Q 40/08; G16H 10/60

(57) ABSTRACT

A healthcare intervention assessment apparatus (10) includes at least one processor (16, 18, 20, 22, 24) programmed to: retrieve data associated with healthcare services provided to patients; build a plurality of utilization vectors representing patients, each utilization vector corresponding to a patient, each utilization vector having vector dimensions representing different types of healthcare services, and each utilization vector being annotated with patient attributes of the patient represented by the utilization vector; scale values of the dimensions of the utilization vectors using scaling factors for a chosen analysis type; perform an analysis of the chosen analysis type on the scaled utilization vectors to determine at least one of a dominant scaled utilization vector, at least one outlier, or at least one range of the scaled values of the dimensions of the utilization vectors; and a display (26) to display a quantitative result of the analysis.

### FIELD

The following relates generally to the medical intervention and selection arts, clinical care decision arts, and related arts.

### BACKGROUND

The Accountable Care Organization (“ACO”) model for providing medical care is increasingly used to tie reimbursement for provided medical care to value metrics for the provided care. The ACO may be used to assess total cost of care for a population, with the goal of reducing this total cost of care by leveraging reimbursements to encourage higher value care pathways.

ACOs have proliferated throughout the United States in the past few years. Understanding, predicting and optimizing service utilization patterns has been identified as a component for ACOs to be successful in shared saving goals.

Various approaches have been employed to analyze healthcare services utilization by patients. For example, in an approach disclosed in Ebadollahi et al., U.S. Pub. No. 2012/0209620, patients are represented by utilization profile vectors. In examples therein, an eight-dimensional utilization profile vector is constructed to represent each patient's yearly utilization, where each dimension records the number of visits of each one of the seven healthcare service types (primary care physician visits, specialist office visits, independent laboratory visits, out-patient hospital visits, in-patient hospital visits, home visits, emergency room/urgent care visits), with one additional “other” category. Clustering analysis is then used to identify dominant or rare utilization patterns, and statistical modelling is performed to link clinical characteristics to utilization patterns.

The following discloses a new and improved systems and methods that address the above referenced issues, and others.

### SUMMARY

In one disclosed aspect, a healthcare intervention assessment apparatus includes at least one processor programmed to: retrieve data associated with healthcare services provided to patients; build a plurality of utilization vectors representing patients, each utilization vector corresponding to a patient, each utilization vector having vector dimensions representing different types of healthcare services, and each utilization vector being annotated with patient attributes of the patient represented by the utilization vector; scale values of the dimensions of the utilization vectors using scaling factors for a chosen analysis type; perform an analysis of the chosen analysis type on the scaled utilization vectors to determine at least one of a dominant scaled utilization vector, at least one outlier, or at least one range of the scaled values of the dimensions of the utilization vectors. A display displays a quantitative result of the analysis.

In another disclosed aspect, a non-transitory storage medium stores instructions that are readable and executable by one or more microprocessors to perform a method. The method includes: retrieving data associated with healthcare services provided to patients; building a plurality of utilization vectors representing patients, each utilization vector corresponding to a patient, each utilization vector having vector dimensions representing different types of healthcare services, and each utilization vector being annotated with patient attributes of the patient represented by the utilization vector; scale values of the dimensions of the utilization vectors using scaling factors for a chosen analysis type; perform an analysis of the chosen analysis type on the scaled utilization vectors to determine at least one of a dominant scaled utilization vector, at least one outlier, or at least one range of the scaled values of the dimensions of the utilization vectors; and displaying the at least one vector.

One advantage resides in determining one or more dominant healthcare interventions with a high cost.

Another advantage resides in analyzing multiple options of healthcare interventions to determine an optimal intervention usage plan.

Another advantage resides in analyzing multiple options of healthcare intervention based on patient benefit and resource allocation

Another advantage resides in providing for comparative analysis of the utilization of different healthcare services having widely differing characteristics (e.g. different costs, different medical staff loading, differing statistical effectiveness).

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a healthcare treatment planning apparatus for determining a healthcare treatment plan as disclosed herein.

FIG. 2 illustrates an exemplary flow chart for a method of use of the apparatus of FIG. 1.

### DETAILED DESCRIPTION

ACOs and health care organizations are in the process of transforming from a volume-to-value based care. One of the important value drivers is reduced utilization. Hospitalization and unnecessary use of healthcare services is a cause of stress and dissatisfaction among patients. A successful approach to transforming to a value based care is to a) understand the population that an ACO is serving, b) identify cost-hot-spots (i.e., utilization patterns that are major cost drivers), c) identify gaps and manage care transitions that are meaningful to patient cohorts and their corresponding utilization pattern.

Analytics is a key enabler of population management, which help with risk-stratification and evidence based approach to enable proactive intervention for per-capita cost reduction. Using advanced analytics, an ACO population can be understood by identifying dominant utilization patterns and provide actionability by assessing the cost and benefit of intervening in patient cohorts to reduce utilization.

As discussed in the background, vector-based analyses of healthcare services utilization is known, for example by representing patients via utilization profile vectors as described in Ebadollahi et al., U.S. Pub. No. 2012/0209620. However, it is recognized herein that such an approach in which diverse healthcare services are mapped to a common vector space is problematic for ACO management tasks, in part because the different healthcare services having widely varying characteristics. For example, a home visit by a traveling nurse has some beneficial characteristics, e.g. limited medical staff usage (a single nurse), and potentially low equipment cost. By contrast, an emergency room visit entails much higher medical staff usage and equipment cost. The term “visit” may also have differing significance for different types of healthcare services—for example a visiting nurse may be typically scheduled to visit on a recurring basis (e.g. once/week) whereas a primary care physician office visit may be expected to occur less frequency (e.g. every six months), and an emergency room visit is preferably a singular event.

To address such problems, in some embodiments disclosed herein, a per-dimensional scaling is applied to the utilization vector to provide comparability of the various healthcare services according to a desired comparison metric. For example, the comparison metric may be staff loading (possibly broken into different staff types, e.g. doctors, nurses, nurse practitioners), or equipment cost, or so forth. With such scaling, the various types of healthcare services are more meaningfully compared. Moreover, different scaling factors can be employed for different types of analyses in order to assess healthcare services utilization in terms of different metrics (staff loading, equipment costs, et cetera).

It is further recognized herein that existing utilization vector-based approaches may fail to account for healthcare services that are functional alternatives. For example, an out-patient hospital visit or a primary care physician office visit may perform similar healthcare functions such as providing routine monitoring of an existing medical condition or addressing a relatively minor medical issue. On the other hand, an emergency room visit usually serves the very different function of providing immediate treatment for an acute condition. In approaches disclosed herein, such healthcare service similarities are represented in the utilization vector model by designating the unit vector direction for similar health care services in the same direction, so that they are additively combined, and possibly using different scaling weights for the different services.

As used herein, the term “dominant” healthcare service (and variants thereof) refers to a healthcare service that has a higher usage rate than other healthcare services (e.g., based on number of patient visits, or cost, or staffing load, resource allocation, or some other usage metric).

As used herein, the term “intervention”, “medical intervention,” or “healthcare service” (and variants thereof) refers to a type of medical treatment event used by a patient (e.g., a primary care physician office visit, an outpatient hospital visit, an emergency department visit, a hospitalization episode, a laboratory test, a pharmacy prescription, and the like). It will be appreciated that the terms “intervention” and healthcare service” can be used interchangeably herein.

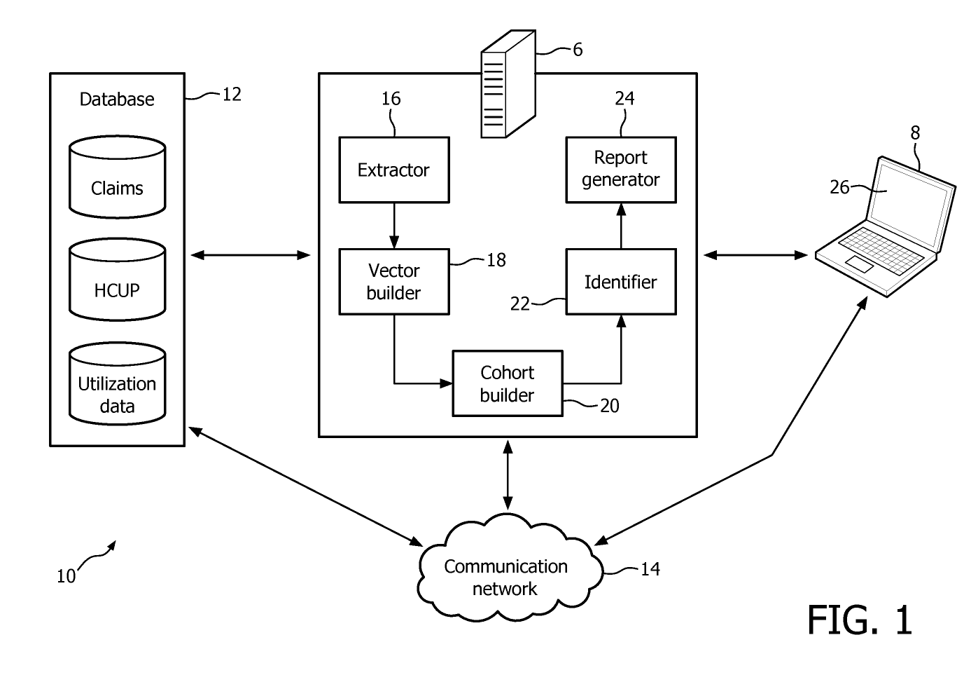

With reference to FIG. 1, an exemplary embodiment of a healthcare intervention assessment apparatus 10 is shown. The apparatus 10 may be implemented on a server computer 6 or other electronic data processing device, and accessed via a user interface device such as an illustrative desktop or notebook computer 8. The apparatus 10 is configured to extract and integrate relevant data needed to determine high usages of one or more medical intervention procedures (e.g., primary care physician (PCP) office visit events, outpatient hospital visit events, emergency department (ED) visit events; hospitalization events; laboratory events, pharmacy events, and the like) for a plurality of patients. For example, the apparatus 10 integrates data from at least one healthcare data sources to provide a comprehensive data on patient population interventions within the hospital and the metrics associated with them (e.g., cost of the intervention, hospital resources including staffing to perform the intervention, benefit and/or outcome of the intervention for the patients, and the like). The apparatus 10 is configured to capture relevant data related to patients and their intervention events, including data related to healthcare claims, Healthcare Cost and Utilization Project (HCUP) data, intervention utilization data, and the like. The apparatus 10 then combines this data for each patient into a utilization vector. A dominant utilization vector is determined by grouping individual patient utilization vectors together, for example using clustering or defining cohorts of similar patients based on shared patient attributes (e.g. same age, same ethnicity, combinations of such attributes, or so forth). By connecting the patient intervention data metrics to the utilization vectors, which are then clustered, grouped, or otherwise organized at the population or cohort level, the apparatus 10 allows the information to be better contextualized and thus better understood. The apparatus 10 can be implemented in a healthcare provider facility (e.g., a hospital, a doctor's office, a medical clinic, and the like) or a non-healthcare provider facility (e.g., a medical insurance company, a third party data collection agency, and the like). Advantageously, the apparatus 10 includes one or more components that: (1) determine one or more dominant healthcare interventions with a high cost, resource allocation, and/or benefit to the patient; and (2) analyze multiple options of healthcare interventions to determine an optimal intervention usage plan.

As shown in FIG. 1, the illustrative apparatus 10 (and more particularly the server computer 6) is in communication with at least one database 12. Typically, an ACO organization has access to claims information of an entire patient population. The databases 12 can include at least one of: a data source including healthcare claims of the patient population; a data source including HCUP data of the patient population; and a data source including utilization data of the patient population. For example, the databases 12 can include information related to the patient population, including diseases, comorbidities, chronic medical conditions, demographics, services utilized during each intervention (e.g., hospital visits, primary care physician (PCP) visits, emergency department (ED) visits, outpatient hospital visits, and the like), information on when claims are made from out of the ACO network; patient attributes (e.g., age, gender, ethnicity, annual income, type of medical intervention, and the like). The server 6 can be physically connected to the databases 12 (i.e., via a USB cable or a cord and a corresponding port, or via a wired Ethernet), or electronically via a wireless communications channel or network 14 (e.g., a wireless network, a local area network, a wide area network, a personal area network, the Internet, an intranet, a customer-supplied IEEE 802.11 wireless network, and the like).

The apparatus 10 includes the computer 6 programmed to perform a data analytic to identify dominant utilization patterns that are driving total cost of the corresponding utilization. To do so, the computer 6 is programmed to implement: an extractor 16 that retrieves patient information from the at least one database 12; a vector builder 18 that creates one or more utilization vectors for each patient from the information retrieved from the at least one database 12; a cohort builder 20 that creates one or more cohorts or other utilization vector groupings based on similarly related vectors; an identifier 22 that determines a dominant vector and/or cohort; and a report generator 24 that generates a report including information associated with managing interventions for at least one patient based on the at least one dominant vector/cohort. The report is displayed on a display component 26 of the user interface 8 to display at least one of the vectors, the cohorts, and the report. However, it will be appreciated that the apparatus 10 can add any desired components, or remove any of the shown components.

The illustrative extractor 16 is programmed to retrieve a plurality of data associated with medical intervention information. For example, the extractor 16 is in communication with the databases 12 via the network 14. In some embodiments, the data coming from multiple data sources can be highly heterogeneous, with different data types, data models, formats and semantics. The extractor 16 is configured to interface the different data sources with one or more homogenous programs to interface with at least one database 12 to extract the plurality of data associated with medical intervention information that includes plurality of possible utilization types. From the extracted data, the extractor 16 can convert the different data into a single format, such as a single document model. The extractor 16 then transmits the retrieved data to the vector builder 18.

The illustrative vector builder 18 is programmed to group the retrieved data for each patient into at least one utilization vector annotated with additional patient information (e.g. demographic information). Each vector corresponds to a single patient and represents that patient's utilization (if any) of various healthcare services. For example, the vector builder 18 is programmed to receive the retrieved patient data from the extractor 16. The vector builder 18 then links different medical intervention events together to form a vector related to each event for each individual patient. To do so, each patient of the ACO patient population is represented by a utilization vector that has: (1) dimensions corresponding to the various possible utilization types; and (2) values for each dimension providing metric of the usage of that utilization type. As to (1), for example, the dimensions/utilization types may include: u₁—PCP office visit events; u₂—outpatient hospital visit events; u₃—ED visit events; u₄—hospitalization events; u₅—laboratory events, u₆—pharmacy events, and so forth. As to (2), for example, dimension u₁ may for example be measured as a count of office visits, u₂ as a count of outpatient hospital visits, u₃ as a count of emergency room visits, u₄ as a count of the total number of days in the hospital, and so forth. In one example, a vector for a patient who has had 7 PCP visit events, 2 outpatient visit events, 5 ED visit events; 0 in-hospital visit events; 0 laboratory events; and 1 pharmacy event can be expressed as 7u₁, 2u₂, 5u₃, 0u₄, 0u₅, 1u₆. In another example, a patient vector can be expressed for whether each setting was utilized (e.g., as 0 for “not utilized” and 1 for “utilized). The vector for a patient who has had 7 PCP visit events, 2 outpatient visit events, 5 ED visit events; 0 in-hospital visit events 0 laboratory events; and 1 pharmacy event can be expressed as 1u₁, 1u₂, 1u₃, 0u₄, 0u₅, 1u₆. This vector can be represented as −1u₁, 2u₂, 5u₃, 0u₄, 0u₅, −1u₆. In another example, the vector can represent year-over-year changes in patient utilization For example, a patient who has, compared to the previous year, one less PCP visit and two more outpatient visits, etc., can be expressed as −1u₁, +2u₂, etc. Alternatively the vector can be an n-tuple of just −1 or +1 to indicate a negative or positive trend in utilization along various dimensions. For example, −1u₁, +1u₂, +1u₃, 0u₄, 0u₅, −1u₆ The vector is also annotated with information about the patient, such as the patient's age, gender, ethnicity, chronic medical conditions, or so forth. These annotations are optionally anonymized using a designated anonymization procedure to ensure that patient privacy is preserved. Once the utilization vectors are constructed, the vector builder 18 transmits the vector data structures to the cohort builder 20; the identifier 22; the report generator 24; or the display 26 for display thereon.

With this utilization vector-based framework, numerous analytics can be performed to support ACO decision-making. To do so, the cohort builder 20 is programmed to create patient cohorts from the vectors so that each patient can be compared against each other (e.g., by their respective vectors). In a typical analysis, a patient cohort is defined based on one or more target patient attributes e.g., age, gender, ethnicity, annual income, type of medical intervention, and the like). The patient cohort definition uses the annotations to the utilization vectors to identify the utilization vectors corresponding to patients of a particular age group, gender, chronic medical condition, or other cohort-defining characteristic(s). The cohort builder 20 groups a plurality of similarly-related vectors (e.g., based on the chosen attributes) in a plurality of cohorts. Once built, the cohorts are transmitted to the identifier 22. The cohorts can also or alternatively be sent to the display 26 for display thereon, for example as a point cloud in an n-dimensional utilization vector space in which each point is the endpoint of one utilization vector of the cohort.

The identifier 22 is programmed to analyze the utilization vectors of a cohort to derive information of interest. For example, the vectors contained in a selected cohort can be clustered to identify the most typical (i.e. dominant) utilization. To do so, the identifier 22 can use at least one suitable clustering algorithm (e.g., an agglomerative hierarchical algorithm, a k-means clustering algorithm, a decision-tree based algorithm, and the like) to identify dominant clustering patterns in an ACO population. For example, if the cohort is found to form a single cluster in the utilization vector space then the centroid of that cluster represents the most common utilization pattern for that cohort. Moreover, the spread of the cluster indicates how variable the healthcare services utilization is amongst the patients of the cohort—a tightly packed cluster indicates that most patients follow the same utilization pattern whereas a broadly spread-out cluster indicates the patients follow a wide range of utilization patterns. This can additionally/alternatively be observed visually by displaying the point cloud of the cluster on the display device 26.

Other patterns may be found automatically by the clustering or visually perceived in a displayed point cloud. For example, if two distinct clusters with different centroids are identified, this indicates that patients of the cohort form two distinct groups of healthcare service utilization patterns. This may lead the ACO employee performing the analysis to consider the patient attributes of the two clusters to determine whether the two distinct clusters may be correlated with different patient characteristics. For example, if it is determined that one of the clusters contains mostly male patients and the other cluster contains mostly female patients, then this indicates a gender-based difference in utilization pattern. In such a case, the ACO employee may choose to re-execute the identifier 22 for a “sub”-cohort of male patients and another “sub”-cohort of female patients to more particularly define the two gender-differentiated clusters.

As another example, the clustering (automated performed by the identifier 22, and/or manual clustering via visualization shown on the display 26) may identify multiple clusters distinguished by economic income level (if this is an attribute labeled to the utilization vectors). Sub-cohorts for different income levels may then be clustered to more precisely define the income level-differentiated clusters. Such an analysis might, for example, reveal relatively heavier usage of emergency room services by lower income patients which may be attributable to lack of medical insurance or lack of a regular PCP. If insurance information is also available as labeled attribute information of the utilization vectors, then a parallel analysis could be performed for the sub-cohort of patients without medical insurance and the sub-cohort of patients with medical insurance (optionally further broken down by type or quality of insurance if this level of information detail is annotated to the utilization vectors) in order to support or contradict the belief that the higher usage of emergency room services by lower income patients is due to lack of medical insurance.

In some cases, the clustering may fail (e.g., by failing to identify a dominant vector). This is still informative for the ACO, as it indicates that the patients making up the analyzed cohort are not receiving well-defined standardized care, perhaps (by way of illustration) providing a statistical indication that patients are not well-informed as to the best choice of healthcare service for a chronic condition defined by the cohort. In other examples, outliers defined as individual patients, or small groups of patients, whose utilization vectors are far away from the cluster center can be identified with further inspection.

In some analyses, the cohort builder 20 may be skipped and all utilization vectors of the population are transmitted directly to the identifier 22. The identifier 22 can perform clustering on the entire population, as described above (in this case the “cohort” is the entire population). Clusters thereby identified in the population can be analyzed to identify common attributes. For example, the patient population management interventions can be evaluated against each cluster of utilization for cost, benefit, and resource allocation (e.g., by comparing the magnitudes of each vector against each other). A dominant vector is indicative of the highest medical intervention cost and benefit information. The dominant vectors can be sent to the display 26 for display thereon.

In some embodiments, the report generator 24 generates a report that includes including information associated with managing interventions for at least one patient based on the at least one dominant vector or cohort. The report generator 24 can also include, in the report, a comparison of the dominant vectors/cohorts against similar vectors/cohorts (i.e., based on the same attributes or utilization types).

As previously noted, each dimension of the utilization vector corresponds to a healthcare service, and the value for each dimension corresponds to a metric of the usage of that healthcare service. As further discussed, in some embodiments the value is in terms of a count of visits to the PCP office, emergency room, or so forth. Some dimensions may not be amenable to a visit count metric, e.g. usage of a pharmacy may be quantifiable in terms of a number of prescriptions filled, or hospital in-patient service may be quantifiable in terms of number of distinct hospitalization event or total number of days in the hospital. It will be appreciated that these various dimensional metrics are not readily comparable, e.g. the “cost” of a PCP office visit is difficult to directly compare with the “cost” of filling a prescription.

To provide comparability between dimensions of the utilization vector, in some embodiments each dimension value is scaled by a scaling factor to provide more meaningful quantitative comparisons. The choice of scaling factor depends upon the initial unit (e.g. visits count, or number of prescriptions filled or so forth) and the desired basis of comparison (e.g., total expenditure on the healthcare service, or staffing load consumed by the service, or so forth). The scaling factors are suitably chosen based on empirical data. For example, if a cost comparison is desired, then the scaling factor for a PCP visit might be the reimbursable cost for a PCP visit on an insurer's reimbursement schedule, while the scaling factor for a pharmacy prescription may be the average reimbursed prescription cost, and the scaling factor for in-patient hospitalization quantified by number of days in the hospital may be the reimbursable cost for one day in the hospital.

With suitable scaling, the magnitude of a patient's utilization vector becomes a (at least approximate) quantitative measure of the total cost of healthcare services consumed by the patient, and the value of each dimension is a (again at least approximate) quantitative measure of the cost of the particular healthcare service represented by that dimension. Ratios of values of different dimensions represent cost ratios for the corresponding healthcare services.

With such scaling, the clustering can provide additional quantitative information. By way of illustration, consider the previous example in which one cluster is for a cohort of uninsured patients and the other cluster is for a cohort of insured patients. In addition to the distinct clusters for these two cohorts indicating distinct healthcare service utilization patterns for the two groups, the magnitude of the centroid vector of each cluster when suitable scaling is used as described above now provides a quantitative measure of the cost of each respective utilization pattern. Thus, if the centroid vector of the cluster of uninsured patients is twice as large in magnitude as the centroid vector of the cluster of insured patients, this is an indication that the higher usage of emergency room services by the uninsured patients is increasing the total cost of providing healthcare services for those patients. While this result may seem intuitive, other similarly obtained results may be counterintuitive. For example, a similar analysis may find that the cluster defined by a cohort of patients receiving an expensive drug from the pharmacy (hence resulting in a large scaled magnitude for the pharmacy dimension of the utilization vectors of this cohort) may nonetheless have a smaller-magnitude cluster centroid vector than a cluster defined by otherwise similar patients who did not receive the expensive drug. Such a result indicates that the drug, although expensive, is apparently effective to the extent that it reduces the overall cost of care for patients receiving that drug. Further analysis of the respective clusters may perhaps indicate that this cost savings is due to a combination of reduced hospitalization costs and reduced PCP visits for the cohort receiving the expensive drug, as indicated by greatly reduced scaled values for those dimensions of the centroid vector of the cluster representing the group receiving the expensive drug.

Other vector magnitudes besides those of the cluster centroids may also be usefully analyzed. For example, the difference between the longest and shortest utilization vectors in a cluster (optionally after discarding any outliers) may be compared to determine the range of costs for healthcare services incurred by the cohort represented by the cluster. A large range may indicate a situation in which total cost can be reduced by modifying ACO procedures to promote utilization patterns corresponding to the shortest vectors.

When the vectors are scaled, the dimensional scaling factors may also optionally be adjusted to perform “what if” scenarios. By way of illustration, in the foregoing example the impact of an increase in the cost of the subject drug may be modeled by increasing the scaling factor for the drug dimension, and this thus provides a way to estimate the price point at which the cost of the drug makes it cost ineffective (although other factors such as quality of life for patients receiving the drug should also be considered in such decision-making). In another example, the scale of the dimensions of the vectors can be adjusted with a staff utilization schedule. Once the scales are adjusted, the adjusted scale of the vector can be simulated to determine at least one of a cost, benefit, and resource allocation of the utilization type of the vectors. For example, if the dimensions are scaled to cost-equivalents using a current reimbursement schedule, contemplated adjustments to the reimbursement schedule can be simulated in the utilization vector space. Similar “what-if” analyses can be performed in conjunction with staff-utilization dimensional scaling to investigate possible staff reallocation. When the scaled value of at least one of the simulated vectors is dominant relative to the original scaled value of the vector, the magnitude of the vector can be adjusted accordingly to the dominant value. The report generator 24 can then send the report to the display 26 for display thereon.

Advantageously, the same set of patient utilization vectors generated by the vector builder 18 can thereby be used to perform materially different ACO decision-making support analyses, by applying appropriate scaling factors. For example, the same set of utilization vectors may be used to perform cost analyses by scaling in accord with costs of providing the respective healthcare services; and also for performing medical staff load analyses by scaling in accord with the staff load consumed by the respective healthcare services. Such analyses can be done so long as the analyzed parameter (e.g. cost, staff load) at least approximately scales with chosen units of the dimensions. This is expected to be the case for the illustrative examples of using a unit of visits for services such as PCP visits, out-patient hospital visits, et cetera, and a unit of number of days in the hospital or number of hospitalization episodes for in-patient hospitalizations services, and so forth.

In leveraging the utilization vectors for such diverse analyses, it may also be the case that some dimensions are either irrelevant (and hence can be removed for the analysis) or may be functionally equivalent and hence combined for the analysis. For example, an out-patient hospital visit or a PCP office visit may perform similar healthcare functions, and a given patient may choose to either visit his or her PCP or make an outpatient hospital visit with similar results (statistically) in either case. In terms of staff load, both options may be equivalent (a single doctor sees the patient in either case) and so the PCP visit and outpatient hospital visit dimensions may be additively combined as a single dimension for staff loading analyses. On the other hand, if the reimbursable cost of an out-patient hospital visit is much higher than the reimbursable cost of a PCP office visit, then these dimensions may be kept as separate dimensions for cost analyses.

It will be appreciated that improved performance of the apparatus 10 can be obtained by appropriate selection and/or combination of utilization vector dimensions and/or scaling of the dimensions to reflect cost, staffing or the like. Advantageously, the assumption that cost correlates with utilization is relaxed, thereby allowing the apparatus to operate to compare vectors and cohorts based on patient benefit and resource allocation, instead of just cost.

The various data processing components 16, 18, 20, 22, and 24 are suitably implemented as a microprocessor, e.g. the CPU of the server 6, programmed by firmware or software to perform the disclosed operations. In some embodiments, the microprocessor is integral to the apparatus 10, so that the data processing is directly performed by the apparatus. In other embodiments the microprocessor is separate from the patient monitoring device 10, for example being the microprocessor of a desktop computer. The various data processing components 16, 18, 20, 22, and 24 of the apparatus 10 may also be implemented as a non-transitory storage medium storing instructions readable and executable by a microprocessor (e.g. as described above) to implement the disclosed operations. The non-transitory storage medium may, for example, comprise a read-only memory (ROM), programmable read-only memory (PROM), flash memory, or other repository of firmware for the apparatus 10. Additionally or alternatively, the non-transitory storage medium may comprise a computer hard drive (suitable for computer-implemented embodiments), an optical disk (e.g. for installation on such a computer), a network server data storage (e.g. RAID array) from which the apparatus 10 or a computer can download the system software or firmware via the Internet or another electronic data network, or so forth.

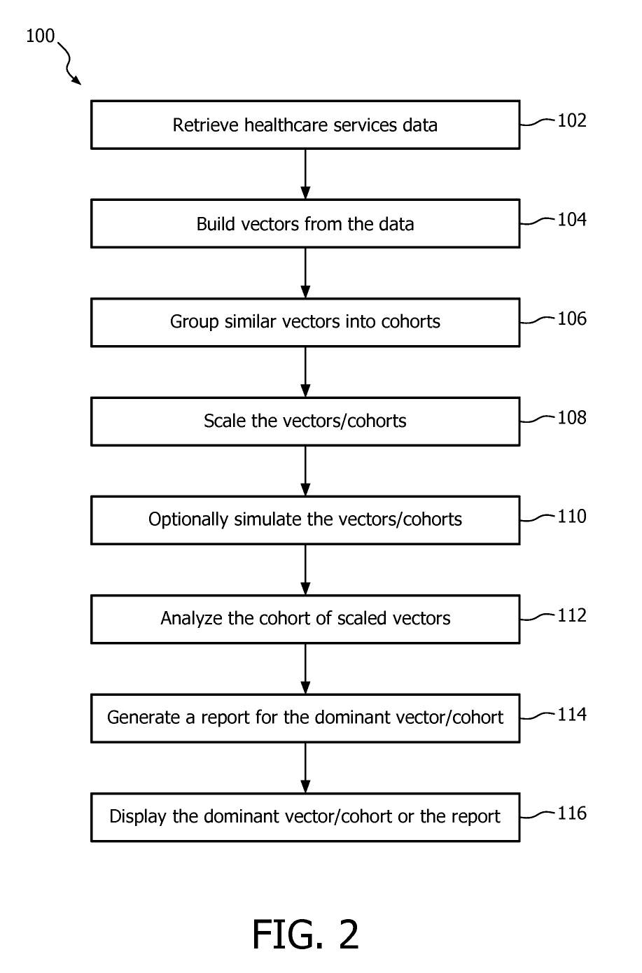

FIG. 2 shows an exemplary flow chart of a method 100 of using the apparatus 10. The method 100 includes: retrieve data associated with healthcare service information provided to patients (102); build a plurality of utilization vectors representing patients in which each utilization vector corresponds to a patient, each utilization vector having vector dimensions representing different types of healthcare services, and each utilization vector being annotated with patient attributes of the patient represented by the utilization vector (104); group the plurality of vectors into a plurality of cohorts based on similarly related vectors having one or more specified patient attributes (106); scaling the vectors with a magnitude indicative of at least one of a cost, a benefit of the utilization type, and a resource allocation of the vector (108); optionally simulating the impact of changed conditions (e.g. increase/decrease in drug price or in hospitalization costs) by adjusting the scaling factors (110); perform an analysis of the chosen analysis type on the scaled utilization vectors of the cohort to determine at least one of a dominant scaled utilization vector, at least one outlier, or at least one range of the scaled values of the dimensions of the utilization vectors (112); generate a report including information associated with managing interventions for at least one patient based on the at least one dominant vector or cohort (114); and display at least one of the vectors, the cohorts, and the report on a display (116).

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A healthcare intervention assessment apparatus, comprising:
at least one processor programmed to:
retrieve data associated with healthcare services provided to patients;
build a plurality of utilization vectors representing patients, each utilization vector corresponding to a patient, each utilization vector having vector dimensions representing different types of healthcare services, and each utilization vector being annotated with patient attributes of the patient represented by the utilization vector;
scale values of the dimensions of the utilization vectors using scaling factors for a chosen analysis type;
perform an analysis of the chosen analysis type on the scaled utilization vectors to determine at least one of a dominant scaled utilization vector, at least one outlier, or at least one range of the scaled values of the dimensions of the utilization vectors; and
a display to display a quantitative result of the analysis.

2. The apparatus of claim 1, wherein the at least one processor is further programmed to:
group the plurality of vectors into at least one cohort based on similarly related vectors having one or more specified patient attributes;
wherein the analysis is performed on the cohort.

3. (canceled)

4. The apparatus according to claim 2, wherein the at least one processor is further programmed to:
select one or more cohorts based on at least one selected patient attribute; and
cluster the utilization vectors of a selected cohort to identify a dominant healthcare service utilized by patients of the cohort.

5. The apparatus according to claim 4, wherein the at least one processor is programmed to:
cluster the utilization vectors by at least one of an agglomerative hierarchical algorithm, a k-means clustering algorithm, and a decision-tree based algorithm.

6. The apparatus according to claim 1, wherein the at least one processor is further programmed to:
generate a report including the dominant scaled utilization vector, outlier, or range of the scaled values information;
wherein the display is configured to display the report.

7. The apparatus according to claim 1, wherein the at least one processor is further programmed to:
interface with at least one database to extract the data associated with medical intervention information, the medical intervention information including a plurality of possible utilization types.

8. The apparatus according to claim 1, wherein at least one of:
the analysis type is a cost analysis and the scaling converts the values of the dimensions to cost values; and the analysis type a resource allocation analysis and the scaling converts to values of the dimensions to resource allocation values.

9. (canceled)

10. The apparatus according to claim 1, wherein the at least one processor is further programmed to:
adjust a scale of the at least one vector to at least one of:
a cost-equivalent using a current patient reimbursement schedule; and
a staff utilization schedule;
simulate the adjusted scale of the vector to determine at least one of a cost, benefit, and resource allocation of the utilization type of the at least one vector; and
adjust the magnitude of the vector when the simulated scaled value is dominant relative to an original scaled value of the vector.

11. A non-transitory storage medium storing instructions readable and executable by one or more microprocessors to perform a method, comprising:
retrieve data associated with healthcare services provided to patients;
build a plurality of utilization vectors representing patients, each utilization vector corresponding to a patient, each utilization vector having vector dimensions representing different types of healthcare services, and each utilization vector being annotated with patient attributes of the patient represented by the utilization vector;
scaling values of the dimensions of the utilization vectors using scaling factors for a chosen analysis type;
performing an analysis of the chosen analysis type on the scaled utilization vectors to determine at least one of a dominant scaled utilization vector, at least one outlier, or at least one range of the scaled values of the dimensions of the utilization vectors; and
displaying the at least one vector.

12. The non-transitory storage medium according to claim 11, wherein the one or more microprocessors are further programmed to:
group the plurality of vectors into at least one cohort based on similarly related vectors having one or more specified patient attributes; and
wherein the analysis is performed on the cohort.

13. (canceled)

14. The non-transitory storage medium according to claim 13, wherein the one or more microprocessors are further programmed to:
select one or more cohorts based on at least one selected patient attribute; and
cluster the utilization vectors of a selected cohort to identify a dominant healthcare service utilized by patients of the cohort.

15. The non-transitory storage medium according to claim 14, wherein the one or more microprocessors are programmed to:
cluster the utilization vectors by at least one of an agglomerative hierarchical algorithm, a k-means clustering algorithm, and a decision-tree based algorithm.

16. The non-transitory storage medium according to claim 11, wherein the one or more microprocessors are further programmed to:
generate a report including the dominant scaled utilization vector, outlier, or range of the scaled values information; and
display the report.

17. The non-transitory storage medium according to claim 11, wherein the one or more microprocessors are further programmed to:
interface with at least one database to extract the plurality of data associated with medical intervention information, the medical intervention information including a plurality of possible utilization types.

18. (canceled)

19. (canceled)

20. The non-transitory storage medium according to claim 11, wherein the one or more microprocessor is further programmed to:
adjust a scale of the at least one vector to at least one of:
a cost-equivalent using a current patient reimbursement schedule; and
a staff utilization schedule;
simulate the adjusted scale of the vector to determine at least one of a cost, benefit, and resource allocation of the utilization type of the at least one vector; and
adjust the magnitude of the vector when the simulated scaled value is dominant relative to an original scaled value of the vector.
